(19) United States
(12) Patent Application Publication
(10) Pub. No.: US 2024/0307575 A1
(43) Pub. Date: Sep. 19, 2024

(54) METHODS FOR PRESERVING PLANT MATTER

(71) Applicant: NOVAGREEN TECHNOLOGIES LTD, Kibbutz Megiddo (IL)

(72) Inventors: Boaz Nitzan, Sde Warburg (IL); Joseph Englander, Petah Tikva (IL)

(73) Assignee: NOVAGREEN TECHNOLOGIES LTD, Kibbutz Megiddo (IL)

(21) Appl. No.: 18/575,861

(22) PCT Filed: Jul. 5, 2022
(86) PCT No.: PCT/IL2022/050717

§ 371 Date: Jan. 1, 2024

Related U.S. Application Data
(60) Provisional application No. 63/218,514, filed on Jul. 6, 2021.

(51) Int. Cl. A61L 2/20; A61L 2/00; A61L 2/10; A61L 2/18

(52) U.S. Cl. CPC A61L 2/208; A61L 2/0023; A61L 2/0047; A61L 2/0088; A61L 2/0094; A61L 2/10; A61L 2/186; A61L 2/202; A61L 2202/21

(57) ABSTRACT

Described herein is an improved method for the decontamination and/or preservation of plant matter, in particular Cannabis plant matter. According to an embodiment, the method for the decontamination of plant matter comprises: removing air from a chamber comprising plant matter; contacting the plant matter with a sterilant while the plant matter is in the chamber, under a pressure lower than atmospheric pressure; reducing pressure by removing gas from the chamber; repeating the contacting and reducing steps at least a second time; and removing the sterilant from the chamber to provide decontaminated plant material.

### CROSS REFERENCE TO RELATED APPLICATIONS

Benefit is claimed to U.S. Provisional Patent Application No. 63/218,514 filed Jul. 6, 2021; the contents of which are incorporated by reference herein in its entirety.

### FIELD

Provided herein are methods for preserving plant matter, in particular matter from the plants of the genus Cannabis.

### BACKGROUND

Cannabis is a genus of plants comprising the species Cannabis sativa, C. indica, and C. ruderalis. Cannabis plants have been cultivated for a variety of uses including making fibers (hemp), medicinal use and recreational drug use. Cannabis is also commonly known as marijuana.

One of the most common ways that cannabis is used for medicinal use in many countries (also known as medical marijuana) is through smoking. Smoking cannabis is typically performed by using a pipe, by using a water-pipe (also known as a bong) which filters the smoke through water before inhalation or by rolling in paper to form marijuana cigarettes, also known colloquially as “joints.” The part of the plant typically used for smoking is the whole flower and budding leaf.

Cannabinoids are compounds active on cannabinoid receptors in humans. Cannabinoids of plant origin, also known as phyto-cannabinoids, are abundant in plants of the Cannabis genus. Two known cannabinoids which are present in relatively high concentrations in various strains of Cannabis sativa are tetrahydracannabinol-acid (THCA) or its decarboxylated product tetrahydracannabinol (THC) and cannabidiolic acid (CBDA) or its decarboxylated product cannabidiol (CBD).

Cannabis inflorescence is typically grown by an authorized grower, packaged, and shipped, through a supply chain, until it reaches an end user. Because cannabis inflorescence is derived from living material, microbes are frequently present within the inflorescence even when cannabis plants are grown under controlled conditions. Such microbes include fungi and bacteria, which have been shown to be harmful to humans when smoking cannabis inflorescence contaminated with the microbes. In order to kill microbes after harvesting cannabis inflorescence and before packaging, irradiation with gamma waves has been used. A disadvantage of such a process is the impact of the irradiation on terpenes present in the cannabis inflorescence, which are known to contribute to the taste and smell profile of the inflorescence. (Hazekamp A (2016) Evaluating the Effects of Gamma-Irradiation for Decontamination of Medicinal Cannabis. Front. Pharmacol. 7:108.)

### SUMMARY

Described herein is an improved method for the decontamination and/or preservation of plant matter, in particular Cannabis plant matter.

According to an embodiment, the method for the decontamination of plant matter comprises: removing air from a chamber comprising plant matter; contacting the plant matter with a sterilant while the plant matter is in the chamber, under a pressure lower than atmospheric pressure; reducing pressure by removing gas from the chamber; repeating the contacting and reducing steps at least a second time; and removing the sterilant from the chamber to provide decontaminated plant material.

Further embodiments relate to a method for the decontamination of plant matter comprising: removing air from a chamber comprising plant matter; contacting the plant matter with a chlorine agent while the plant matter is in the chamber, under a lower pressure that is lower than atmospheric pressure; contacting the plant matter with an aqueous hydrogen peroxide solution while the plant matter is in the chamber, under a higher pressure that is lower than atmospheric pressure, and removing the sterilant from the chamber to provide decontaminated plant material.

The foregoing and other objects, features, and advantages will become more apparent from the following detailed description, which proceeds with reference to the accompanying figures.

### BRIEF DESCRIPTION OF THE DRAWINGS

FIG. 1 is a flow diagram depicting a method for decontaminating plant matter according to an embodiment.

### DETAILED DESCRIPTION

### I. Terms

Unless otherwise noted, technical terms are used according to conventional usage.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

In case of conflict, the present specification, including explanations of terms, will control. In addition, all the materials, methods, and examples are illustrative and not intended to be limiting.

### II. Overview of Several Embodiments

Provided herein are methods for decontamination of plant matter using vacuum, and sterilants.

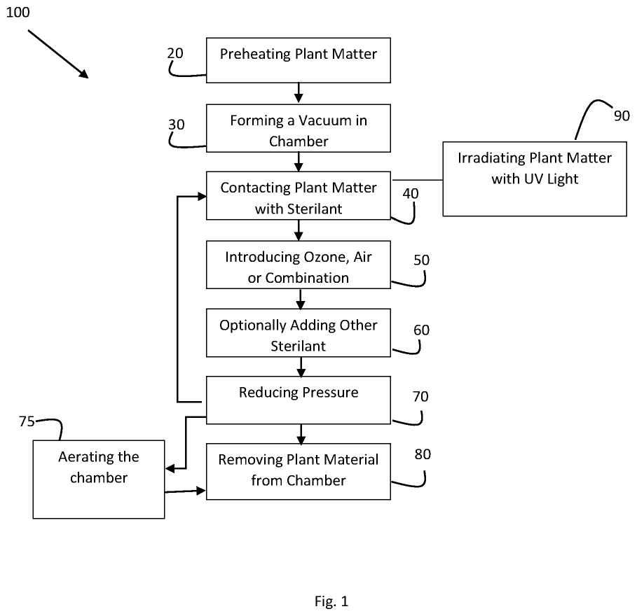

Reference is made to FIG. 1 which depicts a flow diagram showing a method 100 for decontamination of plant matter.

Method 100 comprises block 20, preheating plant matter. The plant matter, according to an embodiment is plant matter from a cannabis plant. Optionally, the plant matter is cannabis inflorescence. Optionally, the preheating may be performed to a temperature of 35° C. to 40° C.

According to an embodiment, the heating is performed in a vacuum chamber. Optionally, the plant matter is vented by removal of air from the chamber after heating.

Method 100 further comprises block 30, forming a vacuum in a chamber, the chamber containing the plant matter. The chamber is a closed chamber which can be kept airtight, under vacuum. Optionally, the vacuum is formed at a pressure of between 0.5 and 1.5 millibar (mbar).

Method 100 further comprises block 40, contacting plant matter with a sterilant while the sterilant is within the vacuum chamber, under vacuum. Optionally, the sterilant is selected from the group consisting of: hydrogen peroxide, ozone, and a chlorine agent. The chlorine agent may comprise hypochlorite ion, hypochlorous acid, Sodium dichloroisocyanurate (NaDCC) or other chlorine containing components which release hypochlorite or hypochlorous acid, upon reaction with water. NaDCC is a chlorinated cyanurate. Other chlorinated cyanurates which may be used include trichloroisocyanuric acid. Optionally, the sterilant is an aqueous mixture of water and hydrogen peroxide, optionally having hydrogen peroxide content of between 15% and 35% by weight.

Preferably, the sterilant should be added while partially maintaining vacuum in the chamber, by maintaining pressure in the chamber less than atmospheric pressure. Preferably, the sterilant is added to the chamber to increase the pressure within the chamber to between 30 and 100 mbar.

Without being bound by theory, it is suggested that the low pressure allows the sterilant added while maintaining low pressure to contact the inner surfaces of the plant material, such as cannabis inflorescence, thereby providing decontamination while maintaining the sterilant in a vapor phase.

Optionally, according to block 40, the sterilant is contacted with the plant matter, at the low pressure, for over 3 minutes. Preferably the sterilant is contacted with the plant matter for less than 15 minutes.

Method 100 further comprises block 50, introducing an additional gas into the vacuum chamber, preferably while maintaining the chamber at a pressure below atmospheric pressure. Optionally, the pressure is between 100 and 700 mbar, preferably 500-600 mbar. Optionally, the additional gas should be contacted with the plant matter at the pressure below atmospheric pressure, for over 3 minutes, preferably for less than 15 minutes.

Optionally, the gas comprises ozone. Optionally, the gas comprises air. Without being bound by theory, it is suggested that ozone added to the chamber will contribute to further decontamination of the plant matter, as ozone will react with the water vapor within the chamber and form more hydrogen peroxide. Similarly, introducing air increases decontamination of the plant matter by forcing/moving the sterilant of block 30 to come into contact with additional surface areas of the plant matter.

Method 100 further comprises block 60, optionally adding an additional sterilant. The sterilant may be the same sterilant used in previous blocks, or may be a different sterilant.

Method 100 further comprises block 70, reducing pressure in the vacuum chamber. The pressure may be reduced to 0.5-1.5 mbar. Optionally, after reducing pressure, the process may return to block 40. The process comprising blocks 40 to block 70 is hereinafter called a “pulse”. Method 100 may comprise 1, 2, 3, or 4 pulses.

Blocks 40-70 may be repeated using the same sterilant as the first pulse, under the same conditions, or different sterilant as the first pulse, under different conditions.

Method 100 further optionally comprises block 75, aerating the chamber. The chamber may be aerated by producing a vacuum in the chamber, then allowing air to flow in, then repeating between 1-5 additional times. This may allow in removal of the sterilant from the plant matter after the plant matter has been treated.

Method 100 further comprises block 80, removing the plant material from the chamber.

Method 100 further comprises block 90, irradiating the plant matter with ultra-violet (UV) light. The UV light can be applied during one or more than one of blocks 40, 50, or 60. The vacuum chamber may be fitted with a UV light to irradiate the plant matter. The UV light may be continuous or in pulses. Optionally, UV-C light having a wavelength of 180-280 nanometers, preferably 240 nm is used. The UV light may be used in any steps of the process in which peroxide is present in the chamber.

Without being bound by theory, it is suggested that UV light, in particular UV-C, increases content of free radicals within the chamber by breaking the O—O bond present in sterilants.

Advantages of the methods described herein are as follows. Irradiation with radioactive material is a method which requires special facilities and precautions and can be costly. Also, irradiation can decrease terpene levels in cannabis plant matter. On the other hand. Methods described herein use readily available solvents and can be effective in small amounts to effectively decontaminate cannabis plant matter from microbial growth, thereby preserving cannabis plant matter, in particular, cannabis inflorescence. The cannabis inflorescence and flower maintain high levels of active cannabinoids and terpenes after the process is performed. Furthermore, the moisture levels in the plant matter are not negatively impact to a greater extent than when using other, known methods.

Challenges associated with decontamination of plant matter, especially cannabis inflorescence, relate to the large surface area of the plant matter. On the one hand, sterilant must penetrate to contact the large surface area to bring about decontamination. On the other hand, it prolonged contact of certain sterilant under certain conditions with plant matter may cause reactions which decompose active ingredients, or aromatic ingredients within the plant matter.

Methods described herein overcome these challenges and allow effective decontamination of inner surfaces while maintaining the active and aromatic components within the plant matter.

According to some embodiments, methods described herein are performed without formation of plasma within the chamber. Plasma, although effective in killing microbes, requires electrodes in close proximity, preferably within 10 mm from each other, in order to effectively form plasma in an industrial setting. The object being decontaminated or disinfected using plasma must have a diameter small enough to fit between the two electrodes to come in contact with the plasma. Methods described herein can be industrially adapted to large amounts of plant matter, and to large pieces of plant matter having dimensions (width, length, height) of greater than 10 mm.

According to an embodiment, described herein is a method for the decontamination of plant matter comprising: removing air from a chamber comprising plant matter; contacting the plant matter with a sterilant while the plant matter is in the chamber, under a pressure lower than atmospheric pressure; reducing pressure by removing gas from the chamber; steps of contacting the plant matter with a sterilant and reducing pressure are performed at least a second time; and removing the sterilant from the chamber to provide decontaminated plant material. Optionally, the plant matter is cannabis inflorescence. Optionally, the air is removed from the chamber of step a to a pressure of below 1.5 mbar. Optionally, contacting the plant matter with a sterilant comprises contacting the plant matter with a primary sterilant at a lower pressure and subsequently contacting the plant matter with a gas or a secondary sterilant at a higher pressure. Optionally, the sterilant comprises an agent selected from the group consisting of: hydrogen peroxide, ozone, and a chlorine agent. Optionally, the chlorine agent is selected from the group consisting of: hypochlorite ion, hypochlorous acid, or a chlorinated cyanurate. Optionally, the chlorinated cyanurate is NaDCC. Optionally, the sterilant is NaDCC 2% by weight. Optionally, the sterilant is a hydrogen peroxide solution having a content of hydrogen peroxide of between 15% and 35% by weight. Optionally, the lower pressure is between 30 and 100 mbar. Optionally, the higher pressure is between 100 and 700 mbar. Optionally, the plant matter is contacted with a primary sterilant at a lower pressure for between 3 and 15 minutes. Optionally, the plant matter is contacted with a gas or a secondary sterilant at a higher pressure for between 3 and 15 minutes. Optionally, removing the sterilant comprises aerating the chamber. Optionally, the microbial load of the plant matter is decreased at least 1000-fold after performing the method. Optionally, the plant matter is heated in the chamber before applying a sterilant. Optionally, the plant matter is heated to a temperature of between 35° C. and 60° C. while the plant matter is contacted with the sterilant. Optionally, the plant matter is irradiated with UV light while contacted with a sterilant. Optionally, steps of contacting sterilant with plant matter and reducing pressure are repeated twice, three times, or four times. Optionally, the steps repeated three times. Optionally, the gas comprises air.

Further described herein is a method for the decontamination of plant matter comprising: removing air from a chamber comprising plant matter; contacting the plant matter with a chlorine agent while the plant matter is in the chamber, under a lower pressure that is lower than atmospheric pressure; contacting the plant matter with an aqueous hydrogen peroxide solution while the plant matter is in the chamber, under a higher pressure that is lower than atmospheric pressure, and removing the sterilant from the chamber to provide decontaminated plant material. Optionally, the plant matter is cannabis inflorescence. Optionally, the air is removed from the chamber to a pressure of below 1.5 mbar. Optionally, the chlorine agent is selected from the group consisting of: hypochlorite ion, hypochlorous acid, or a chlorinated cyanurate. Optionally, the chlorinated cyanurate is NaDCC. Optionally, the sterilant is NaDCC 2% by weight. Optionally, the hydrogen peroxide solution has a content of hydrogen peroxide of between 15% and 35% by weight. Optionally, the lower pressure is between 30 and 100 mbar. Optionally, the higher pressure is between 100 and 700 mbar. Optionally, the plant matter is contacted with a chlorine agent at a lower pressure for between 3 and 15 minutes. Optionally, the plant matter is contacted with a hydrogen peroxide solution at a higher pressure for between 3 and 15 minutes. Optionally, the method comprises a further comprising the step of aerating the chamber. Optionally, the microbial load of the plant matter is decreased at least 1000-fold after performing the method. Optionally, the plant matter is heated in the chamber before performing the method. Optionally, the plant matter is heated to a temperature of between 35° C. and 60° C. while in the plant matter is in contact with the sterilant. Optionally, the plant matter is irradiated with UV light while contacted with a sterilant. Optionally, the chamber is evacuated after contacting the plant matter with an aqueous hydrogen peroxide solution. Optionally, the chamber is evacuated to a pressure of below 1.5 mbar. Optionally, after evacuation, the plant matter is treated to a second cycle of contacting with a chlorine agent and an aqueous hydrogen peroxide solution. Optionally, the plant matter is treated to a third or fourth cycle. Optionally, plasma is not formed in the chamber. Optionally, the plant matter has a length, width, and height each greater than 10 mm.

Some embodiments are described with reference to the examples below.

### EXAMPLES

### Example 1

Samples of cannabis inflorescence in an amount of 200 grams per sample were introduced into a vacuum chamber of 30 liters, pressure was reduced to 10 mbar and various treatments using hydrogen peroxide solution (50% in water) at various temperatures were performed.

A control sample was used, which was not treated, to show microbial load before treatment. The results of the analysis of microbial count is expressed in terms of total count in colony forming units per milliliter (cfu/ml) and yeast and mold count (Y&M) in Table 1 below.

TABLE 1

| | | | Y&M |
|---|---|---|---|
| Sample | TREATMENT | TC (cfu/ml) | (cfu/ml) |
| Control | AVERAGE CONTROL | 208,800 | 20,800 |
| 1 | 30° C. 40 ml | 31,000 | 2,600 |
| 2 | 30° C. 40 ml | 31,000 | 650 |
| 3 | 40° C. 40 ml | 15,000 | 750 |
| 4 | 40° C. 40 ml | 10,000 | 1,200 |
| 5 | 40° C. 40 ml + 1 m + Vent700 | 12,000 | 1,100 |
| 6 | 40° C. 40 ml + 1 m + Vent700 | 19,000 | 8,500 |
| 7 | 40° C. 40 ml + 1 m | 5,400 | 2,600 |
| 8 | 40° C. 40 ml + 1 m | 58,000 | 2,500 |

In each of the trials above, the H₂O₂ solution was introduced over a period of 3 minutes until a pressure of 70-100 mbar, by introduction of 4 injections, each injection of 10 ml. In those indicating “1 m”, the pressure was held for one minute after introduction of hydrogen peroxide. Some of the batches indicating “vent 700” were slowly vented to 700 mbar with air.

The decontamination process appeared to be more effective at 40° C. relative to 30° C. While these conditions were effective in reducing some of the microbial load, the microbial load was above the regulatory requirement of 20,000 cfu/ml for total count and 2000 cfu/ml for yeasts & molds in most of the test results. Furthermore, the test results did not appear to provide consistent results in a reproducible way.

### Example 2

Samples of cannabis inflorescence weighing 200 g each were introduced into a vacuum chamber, pressure was reduced to 1 mbar and various treatments using hydrogen peroxide (HPO) and ozone at various temperatures were performed. The hydrogen peroxide was introduced over 3 minutes, then the pressure was held at 75 mbar for 6 minutes. Ozone was introduced to 500 mbar over 3 minutes and the pressure was held for an additional 6 minutes. In the samples indicated “two pulse,” after a first ozone introduction and holding for 6 minutes, the chamber was then evacuated and a second identical pulse of hydrogen peroxide and ozone was performed. The results of microbial load for the inflorescence and for a control batch are detailed in Table 2.

TABLE 2

| | | | Y&M |
|---|---|---|---|
| Sample | TREATMENT | T.C (cfu/ml) | (cfu/ml) |
| Control | AVERAGE CONTROL | 1,515,000 | 21,500 |
| 1 | 35 ml of HPO to 75 mbar + | 130,000 | 4,000 |
| | O₃ to 500 mbar at 30° C. | | |
| | one pulse | | |
| 2 | 35 ml of HPO to 75 mbar + | 260,000 | 7300 |
| | O₃ to 500 mbar at 40° C. | | |
| | one pulse | | |
| 3 | 35 ml of HPO to 75 mbar + | 6700 | 375 |
| | O₃ to 500 mbar at 30° C. | | |
| | two pulse | | |
| 4 | 35 ml of HPO to 75 mbar + | 12,000 | 570 |
| | O₃ to 500 mbar at 40° C. | | |
| | two pulse | | |

Whereas one pulse reduces the microbial load by about one order of magnitude, the pathogen levels are still high and above the regulatory threshold (20,000 cfu/ml for total count and 2000 cfu/ml for yeasts & molds). Application of two successive pulses on the same cannabis lot, significantly lowers the pathogen load well below the regulatory threshold.

Shortening the treatment time has the additional benefit of reducing the impact of the decontamination cycle on the cannabis buds, and improving the relative humidity of the flowers post treatment.

The following table (Table 3) demonstrates the disinfection abilities of the current apparatus setup and the additive effects of combining different sterilant (hydrogen peroxide and ozone) and the disinfection capability of one pulse versus two pulse process. In table 3, the samples were all preheated to, 40° C. Some of the samples, indicated “O₃ preheat” were preheated in the presence of ozone at atmospheric pressure. Vacuum of 1 mbar was initiated, then hydrogen peroxide was added as the first sterilant, over 3 minutes, then held at 75 mbar for 6 minutes (per pulse). Air was subsequently introduced to a pressure of 500 mbar. Then the chamber was evacuated. For those samples in which “two pulse” is indicated, the process of hydrogen peroxide addition and air addition was repeated as in the first pulse.

TABLE 3

| | | Sample | | Y&M |
|---|---|---|---|---|
| Sample | TREATMENT | weight | TC (cfu/ml) | (cfu/ml) |
| Control | AVERAGE CONTROL | NA | 130,000 | 1,950 |
| 1 | HPO to 75 mbar + air to 500 | 100 gr | 11,000 | 100 |
| | mbar 40° C. one pulse | | | |
| 2 | O₃ preheat, HPO to 75 mbar + | 100 gr | 3,800 | 20 |
| | air to 500 mbar 40° C. one | | | |
| | pulse | | | |
| 3 | HPO to 75 mbar + air to 500 | 300 gr | 48,000 | 30 |
| | mbar 40° C. one pulse | | | |
| 4 | HPO to 75 mbar + O₃ to 500 | 300 gr | 5,200 | 20 |
| | mbar 40° C. two pulse | | | |
| 5 | O₃ preheat, HPO to 75 mbar, | 300 gr | 55,000 | 80 |
| | air to 500 mbar 40° C. one | | | |
| | pulse | | | |
| 6 | O₃ preheat, HPO to 75 mbar, | 300 gr | 300 | 20 |
| | air to 500 mbar 40° C. two | | | |
| | pulse | | | |

These results show that preheating with ozone positively impacts decontamination. For example, when comparing sample 2 to sample 1, it is evident that ozone preheating decreases microbial count in sample 2 relative to sample 1. Furthermore, two pulses are much more effective than using one pulse as seen when comparing sample 6 to sample 5.

### Example 3

Samples of cannabis inflorescence (200 mg) were introduced into a vacuum chamber pressure was reduced to 1 mbar and various treatments using hydrogen peroxide (HPO) and air. For two of the samples, no preheating was performed, and for two of the samples, preheating was performed. Preheating involved warming the plant matter in the chamber at 40° C. for 5 minutes. Either one or two pulses of sterilant were performed. All pulses were performed at 40°. Before each pulse, the pressure in the chamber was reduced to 1 mbar. Then hydrogen peroxide was added to the chamber to a pressure of 60 mbar and was maintained for 6 minutes.

Then, air was added and pressure was maintained at 500 mbar for 6 minutes. The results are shown in Table 4 below.

TABLE 4

| | | Total | | Yeast & |
|---|---|---|---|---|
| | Total | Count | Yeast & | Mold |
| | Count | Fold- | Mold | Fold- |
| Treatment | (cfu/ml) | Reduction | (cfu/ml) | Reduction |
| Control | 1,350,000 | | 1,150,000 | |
| No preheat, one pulse | 5,556 | 243 | 19,828 | 58 |
| No preheat, two pulses | 3,082 | 438 | 3,249 | 354 |
| Preheat, one pulse | 2,316 | 583 | 3,433 | 335 |
| Preheat, two pulses | 1,028 | 1,313 | 415 | 2,770 |

This table demonstrates the synergistic effect of pre-heating and number of pulses of HPO. It can be seen that there is an advantage of two pulses over one pulse in both processes, whether the cannabis inflorescence was preheated or was not pre-heated. When combining both a pre-heating stage and two sterilant pulses a significant effect is achieved showing a decontamination of over 3 orders of magnitude in reduction of microbes.

### Example 4

In addition to ozone and hydrogen peroxide, decontamination using active chlorine species, such as Sodium dichloroisocyanurate (NaDCC), was attempted. NaDCC, the sodium salt of a chlorinated hydroxytriazine, is used as a source of free available chlorine (in the form of hypochlorous acid, HOCl).

Aqueous solutions of NaDCC at variating concentrations from 1% [w/w] up to 5% [w/w] were prepared. The NaDCC solutions were introduced to the disinfection chamber in similar manner to the hydrogen peroxide pulses described above. As shown in the table below, a good correlation was observed between the concentration of the chlorine sterilant and its disinfecting capability. However, at 2% [w/w] an optimal result can be observed. A two-pulse process was superior to a one pulse process using this sterilant. Table 5 describes various sterilants and their effects:

TABLE 5

| | | Total | Yeast & | |
|---|---|---|---|---|
| | | pathogen | Molds | Fold- |
| Sample | Sterilant concentration | Count | Count | Reduction |
| Control | 0 | 490,000 | 152,000 | |
| 1 | 50% H2O2, 2 pulses | 8,033 | 2,492 | 61 |
| 2 | 1%; 1 pulse | 35,100 | 10,857 | 14 |
| 3 | 1%; 2 pulses | 17,900 | 5,553 | 27 |
| 4 | 2%; 1 pulse | 24,500 | 7,600 | 20 |
| 5 | 2%; 2 pulses | 11,075 | 3,436 | 44 |
| 6 | 5%; 1pulse | 32,667 | 10,133 | 15 |
| 7 | 5%; 2 pulses | 40,833 | 12,667 | 12 |
| 8 | 2%; 1 pulse + 50% H2O2, | 5,568 | 1,727 | 88 |
| | 2 pulses | | | |
| 9 | 2%; 2 pulses + 50% H2O2, | 5,698 | 1,767 | 86 |
| | 2 pulses | | | |
| 10 | 5%; 1 pulse + 50% H2O2, | 5,904 | 1,831 | 83 |
| | 2 pulses | | | |
| 11 | 5%, 2 pulse + 50% H2O2, | 3,267 | 1,013 | 150 |
| | 2 pulses | | | |

Each pulse with disinfectant consisted of vacuum to 1 mbar, then introduction of a sterilant to 100 mbar, 6 minutes of keeping the sterilant at that pressure, introduction of air to 500 mbar, then vacuum.

Best disinfecting results were obtained combining more than one sterilant in the process, and the combination of H₂O₂ and NaDCC (sample 8-11) exhibited significant reduction in the microbial load when compared to the same process without the H₂O₂ preliminary pulse (sample 2-7) and when compared to two (2) pulses of H₂O₂ only.

These results demonstrate the advantage of combining more than one type of sterilant in the disinfection process. Similarly, further processes may comprise three different sterilants, for example, a pulse that comprises NaDCC, a second pulse of hydrogen peroxide, and a third pulse of a sterilant such as ozone.

According to an embodiment, the process comprises at least two sterilants in a pulse, the first sterilant being NaDCC and the second sterilant being H₂O₂ in aqueous solution. Without being bound by theory, it is suggested that NaDCC oxidizes organic pathogen in the plant matter, and kills them. There remains residual free chlorine which then reacts with water upon introduction of H₂O₂ in aqueous solution to form hypochlorite in situ, on the surface of the plant matter, which provides further decontamination. The NaDCC. is removed because of excess of water in the reaction when 202 is added and as a result, no residual NaDCC remains on the plant matter.

### Example 5: Analysis of Cannabinoid Content

Cannabis plant matter from various growers was analyzed before, and after various disinfection processes described herein. The results are shown in table 6 and are expressed in percent by weight.

TABLE 6

| | | | | Change in | |
|---|---|---|---|---|---|
| | | Total | Total | Cannabinoids | Change |
| | | Cannabinoids | Cannabinoids | (% by | in |
| Batch | Process | Before | After | weight) | Humidity |
| 1, | H₂O₂ then Ozone, | 16.159 | 17.819 | 1.660 | −2.75 |
| CBD | 40° C. Two 15 | | | | |
| rich | minute pulses. | | | | |
| 2 | H₂O₂ then Ozone, | 17.970 | 19.060 | 1.090 | −2.9 |
| THC | 40° C. Two 10 | | | | |
| rich | minute pulses. | | | | |
| 3 | H₂O₂ then Ozone, | 11.097 | 12.832 | 1.735 | −2.05 |
| | 40° C. Two 10 | | | | |
| | minute pulses, (Total | | | | |
| | THC) | | | | |
| 3 | H₂O₂ then Ozone, | 0.336 | 0.599 | 0.262 | −2.05 |
| | 40° C. Two 10 | | | | |
| | minute pulses (Total | | | | |
| | CBD) | | | | |
| 4 | H₂O₂. Two 10 | 11.097 | 13.018 | 1.921 | −2.25 |
| | minute pulses, with | | | | |
| | UV Irradiation, 1 Hz | | | | |
| | for 10 seconds every | | | | |
| | 30 seconds while | | | | |
| | exposed to sterilant | | | | |
| | (Total THC) | | | | |
| 5 | H₂O₂. Two 10 | 12.505 | 13.603 | 1.098 | −2.36 |
| | minute pulses, with | | | | |
| | UV Irradiation, 1 Hz | | | | |
| | for 5 seconds every | | | | |
| | 10 seconds while | | | | |
| | exposed to sterilant | | | | |
| | (Total THC) | | | | |
| 6 | H₂O₂ then Ozone, | 14.266 | 14.542 | 0.276 | −0.68 |
| THC | 42° C. Two 20 | | | | |
| rich | minute pulses (Total | | | | |
| | THC) | | | | |
| 7 | H₂O₂ then Ozone, | 16.159 | 17.819 | 1.660 | −2.75 |
| CBD | 40° C. Two 12 | | | | |
| rich | minute pulses. | | | | |

The results in Table indicate that different cannabis plant matter batches rom different growers can be sterilized using processes described herein. In all of the processes described, there is a 2-3% moisture loss in the plant matter, which is typical for sterilization processes. All of the processes show that there is little to no degradation of cannabinoids in the plant matter. Variations in the sterilant used, process time and presence or absence of UV did not negatively impact the cannabinoid content. These examples indicate that the processes described herein are robust and can be used for various types of cannabis plant matter, to provide disinfection without negatively impacting the quality of the treated cannabis plant matter.

### Example 5: Inoculation with Various Microbes (Pathogens), and Subsequent Treatment

In this example, cannabis inflorescence (200 mg) was thoroughly sterilized, then inoculated by soaking in 30 ml of microbial suspension, using each of the microbes in the amounts described in table 7 below, including a microbe mix, consisting of a mixture of all 4 microbes in the table.

TABLE 7

| | | Concentration (Colony |
|---|---|---|
| Microbe (Pathogen) | Type | forming units/ml) |
| Candida albicans | Yeast | 10⁵ |
| Aspergillus brasiliensis | Mold | 10⁵ |
| Pseudomonas aeruginosa | Bacteria | 10⁵ |
| E. Coli | Bacteria | 10⁵ |
| Microbe mix (all 4) | Mixture | 10⁶ of each |

After soaking each sample of inflorescence in the respective microbial suspension, each sample was air dried in a laminar flow dryer overnight for 18 hours and refrigerated until sterilization was initiated. Inflorescence was sterilized 24, 48 and 72 hours post inoculation, designated batch 1, batch 2 and batch 3, respectively. Microbial load analysis was performed after each sterilization day. Samples were also inoculated, and not sterilized, to serve as controls. A saline solution was applied to some of the samples instead of a microbial suspension, to serve as a control.

Analysis of microbial count was performed to samples before and after sterilization, and each microbe count was determined using sub-samples of 10 grams each from each sample, and was performed in triplicate. The sub-samples were ground in 20 ml of saline solution, seeded in a petri dish with agar, which were then incubated at 37° C. for 48 hours, for counting of colonies, to determine number of colony forming units (CFU) per cannabis sample. Logarithmic reduction of pathogen as compared to non-sterilized sample was determined.

Sterilization was performed using the following pulse structure:

Samples of cannabis inflorescence (200 mg) were introduced into a vacuum chamber in either a top tray or a middle tray. The top tray was located in the uppermost location in the chamber, where the highest temperature is measured. The temperature was set to be maintained at 50° C., however, due to the fact that heat rises within the chamber, and that sterilant solution was introduced to the chamber as a vapor having a temperature of about 140-150° C., in some of the experiments the temperature in the uppermost chamber reached 54° C.±1° C. and in some 58° C.±2° C. Two or three pulses of disinfection were performed for each sample, and the results are described in Table 8. In each pulse, pressure was reduced to 3±2 mbar. Vapor formed from 100 ml of 15% [w/w] hydrogen peroxide, was added to the chamber to a pressure of 60-100 mbar and was maintained within the chamber for 6 minutes at that pressure. Then, air was added and pressure was maintained at 500 mbar for 6 minutes at that pressure. The air was then removed from the chamber and the pressure reduced to 3±2 mbar. This process constitutes one pulse. One or two additional pulses were performed for samples, for a total of two pulses per sample, or three pulses per sample. In the final pulse, after initiating a vacuum, air was then introduced and the chamber was depressurized to a pressure of 1 atmosphere.

For each pathogen, mean log reduction was calculated by log₁₀ transformation of CFU values, analyzing each microbial treatment process separately. Sterilization processes were compared with a 1-way ANOVA on log transformed data. As each microbe behaved differently over time after inoculation, as will be discussed below, results are presented in Table 8 below as a mean log reduction, which represent the mean of log reduction of batches 1-3 for each microbe. As noted in the column on the right side of the table, a negative control in which 0 pulses were administered, no log reduction of microbial count was shown.

TABLE 8

Number of pulses

| | 2 | 3 | 2 | 3 | 0 |
|---|---|---|---|---|---|
| Tray | Top | Top | Middle | Middle | N/A |
| Aspergillus | 3.3 | 3.3 | 2.0 | 2.2 | 0.0 |
| Candida | 6.3 | 6.3 | 2.8 | 6.0 | 0.0 |
| E. Coli | 3.5 | 4.8 | 3.5 | 4.3 | 0.0 |
| Microbe Mix | 5.5 | 7.0 | 2.8 | 5.1 | 0.0 |
| Psuedomonas | 5.3 | 7.2 | 3.7 | 4.7 | 0.0 |

In the batches inoculated with Aspergillus, a natural decline in microbial count from 24 to 72 hours after inoculation, without treatment, was seen. At 24 hours post inoculation, a 5 log reduction in microbial count was seen after treatment. The middle tray still showed reduction, but less reduction than the top tray, indicating that there is lack of uniformity in conditions in the upper tray. Nevertheless, all disinfection processes were successful.

In the batches inoculated with Candida, a slight increase in microbial count from 24 to 72 hours after inoculation without treatment was seen. The effects of sterilization were extremely noticeable, as in the top tray, both 2 and 3 pulses led to a 6+ fold reduction. In the middle tray, effects of treatment with 2 pulses were shown but were less noticeable, but a 3-pulse treatment was more effective.

In the batches inoculated with E. Coli, a slight increase in microbial count from 24 to 72 hours after inoculation without treatment was seen. Three pulses showed a greater reduction in microbial count than two pulses, in both the top and middle trays.

In batches inoculated with pseudomonas, the microbial count stays relatively consistent from 24 to 72 hours after inoculation, without treatment. Here the top tray, using 2 or 3 pulses, show the highest reduction. Three pulses in the middle tray leads to a similar reduction in microbial count as two pulses in the top tray. The best results were seen in the top tray, using three pulses.

In batches inoculated with the microbe mix, the microbial count stays relatively consistent from 24 to 72 hours after inoculation, without treatment. Best results of are seen in the top tray, with 3 pulses. Very good results are seen on the top tray with two pulses and middle tray with three pulses. The least reduction is seen in the middle tray with one pulse.

In summary, the processes described herein lead to a substantial reduction in microbial load of potentially pathogenic bacteria, and mold using small amounts of sterilant. The processes are sufficient to disinfect plant matter such as cannabis inflorescence, for example, while maintaining desired qualities of the plant matter after the disinfection process.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A method for the decontamination of plant matter comprising:
a. removing air from a chamber comprising plant matter;
b. contacting the plant matter with a sterilant while the plant matter is in the chamber, under a pressure lower than atmospheric pressure;
c. reducing pressure by removing gas from the chamber;
d. repeating steps b. and c. at least a second time; and
e. removing the sterilant from the chamber to provide decontaminated plant material, wherein the plant matter is cannabis inflorescence.

2. (canceled)

3. The method according to claim 1, wherein the air is removed from the chamber of step a to a pressure of below 1.5 mbar.

4. The method according to claim 1 wherein contacting the plant matter with a sterilant of step b comprises contacting the plant matter with a primary sterilant at a lower pressure and subsequently contacting the plant matter with a gas or a secondary sterilant at a higher pressure.

5. The method according to claim 1 wherein the sterilant comprises an agent selected from the group consisting of: hydrogen peroxide, ozone, and a chlorine agent.

6. The method according to claim 5 wherein the chlorine agent is selected from the group consisting of: hypochlorite ion, hypochlorous acid, or a chlorinated cyanurate.

7. The method according to claim 6 wherein the chlorinated cyanurate is NaDCC.

8. The method according to claim 7 wherein the sterilant is NaDCC 2% by weight.

9. The method according to claim 5 wherein the sterilant is a hydrogen peroxide solution having a content of hydrogen peroxide of between 15% and 35% by weight.

10. The method according to claim 4 wherein the lower pressure is between 30 and 100 mbar.

11. The method according to claim 4 wherein the higher pressure is between 100 and 700 mbar.

12. The method according to claim 1 wherein the plant matter is contacted with a primary sterilant at a lower pressure for between 3 and 15 minutes.

13. The method according to claim 1 wherein the plant matter is contacted with a gas or a secondary sterilant at a higher pressure for between 3 and 15 minutes.

14. The method according to claim 1 wherein step e comprises aerating the chamber.

15. The method according to claim 1 wherein the microbial load of the plant matter is decreased at least 1000-fold after performing the method.

16. The method according to claim 1, wherein the plant matter is heated in the chamber before step a.

17. The method according to claim 1 wherein the plant matter is heated to a temperature of between 35° C. and 60° C. while the plant matter is contacted with the sterilant.

18. The method according to claim 1 wherein the plant matter is irradiated with UV light while contacted with a sterilant.

19. The method according to claim 1 wherein steps b. and c. are repeated twice, three times, or four times.

20. The method according to claim 19 wherein steps b. and c. are repeated three times.

21. The method according to claim 4 wherein the gas comprises air.

22.-43. (canceled)
